(19) United States
(12) Patent Application Publication
(10) Pub. No.: US 2025/0336066 A1
(43) Pub. Date: Oct. 30, 2025

(54) NON-INVASIVE PANCREATICOBILIARY DUCT HYPERTENSION ASSESSMENT

(71) Applicant: THE JOHNS HOPKINS UNIVERSITY, Baltimore, MD (US)

(72) Inventors: Venkata S. AKSHINTALA, Towson, MD (US); Rajat MITTAL, Vienna, VA (US); Jung Hee SEO, Baltimore, MD (US); Atif ZAHEER, Baltimore, MD (US); Mouen A. KHASHAB, Towson, MD (US); Vikesh K. SINGH, Ellicott City, MD (US)

(21) Appl. No.: 19/189,498

(22) Filed: Apr. 25, 2025

Related U.S. Application Data
(60) Provisional application No. 63/638,626, filed on Apr. 25, 2024.

(51) Int. Cl. G06T 7/00; A61B 5/00; A61B 5/055; G06T 5/70; G06T 7/11; G06T 7/60

(52) U.S. Cl. CPC G06T 7/0012; A61B 5/004; A61B 5/055; A61B 5/425; G06T 5/70; G06T 7/11; G06T 7/60; G06T 2200/04; G06T 2207/10088; G06T 2207/30092

(57) ABSTRACT

Techniques for non-invasively assessing pancreaticobiliary duct hypertension in a patient are presented. The techniques include: obtaining a three-dimensional image of the patient's pancreaticobiliary duct; segmenting the three-dimensional image of the patient's pancreaticobiliary duct, from which a pancreaticobiliary duct geometry is obtained; specifying at least one flow rate value for the patient; computing, based on the at least one flow rate value and the pancreaticobiliary duct geometry, a relative pressure distribution along the pancreaticobiliary duct geometry; determining, based at least on the relative pressure distribution, a pancreaticobiliary duct hypertension quantification for the patient; and providing the pancreaticobiliary duct hypertension quantification for the patient.

### RELATED APPLICATION

This application claims the benefit of U.S. Provisional Patent Application No. 63/638,626, entitled “Non-Invasive Pancreaticobiliary Duct Hypertension Assessment,” filed Apr. 25, 2024, which is hereby incorporated by reference in its entirety.

### FIELD

This disclosure relates generally to pancreaticobiliary duct hypertension assessment.

### BACKGROUND

Chronic pancreatitis affects approximately 40 per 100,000 individuals in the U.S. Annually, chronic pancreatitis accounts for around 327,000 hospitalizations and 200,000 emergency room visits, costing 2.5 billion dollars. The majority of patients with chronic pancreatitis develop debilitating chronic abdominal pain suspected to be from increased pancreatic ductal pressure as a result of obstructive pathologies. Alleviation of this ductal obstruction through invasive procedures including both endoscopy and surgery has been the cornerstone of managing painful chronic pancreatitis for nearly five decades. The pain relief rates from these invasive procedures are inconsistent and often suboptimal.

In general, obstruction of a pancreaticobiliary duct (e.g., a bile duct or pancreatic duct) can cause abdominal pain. However, for some pathologies that cause pain, for example, it is unclear if the bile duct and pancreatic duct are truly obstructed and therefore if relieving the obstruction will benefit the patient or not. While endotherapy and surgery can relieve PD hypertension (PDH), they have little effect on neuropathy. The current standard of care in these situations includes invasive interventions such as endoscopic retrograde cholangiopancreatography (ERCP) with stent placement or surgery to relieve the obstruction, often with variable results, and with no benefit, if there is indeed no real obstruction. For example, while endotherapy and surgery can relieve pancreatic duct hypertension, they have little effect on neuropathic pancreatitis. Many patients, therefore, undergo unnecessary interventions which may actually increase harm often due to the risk of complications associated with these procedures.

Currently, no noninvasive technology exists to accurately measure bile duct or pancreatic duct pressure.

### SUMMARY

According to various embodiments, a method of non-invasively assessing pancreaticobiliary duct hypertension is presented. The method includes: obtaining a three-dimensional image of a patient's pancreaticobiliary duct; segmenting the three-dimensional image of the patient's pancreaticobiliary duct, from which a pancreaticobiliary duct geometry is obtained; specifying at least one flow rate value for the patient; simulating, based on the at least one flow rate value and the pancreaticobiliary duct geometry, a relative pressure distribution along the pancreaticobiliary duct geometry; determining, based at least on the relative pressure distribution, a pancreaticobiliary duct hypertension quantification for the patient; and providing the pancreaticobiliary duct hypertension quantification for the patient.

Various optional features of the above method embodiments include the following. The pancreaticobiliary duct geometry may include a pancreatic duct geometry, the flow rate value may include a pancreatic juice flow rate value, and the pancreaticobiliary duct hypertension quantification may include a pancreatic duct hypertension quantification. The method may include predicting, based on the pancreatic duct hypertension quantification for the patient, a patient response to pancreatic duct hypertension relief. The method may include performing one of pancreatic duct endotherapy or pancreatic duct surgery on the patient based on the predicting. The three-dimensional image may include an MRI image. The pancreaticobiliary duct hypertension quantification for the patient may include an estimated pressure difference between two locations within the patient's pancreaticobiliary duct. The pancreaticobiliary duct hypertension quantification for the patient may include an estimated pancreaticobiliary duct resistance. The determining may include performing a regression for a plurality of flow rate values and a corresponding plurality of pressure difference values for the pancreaticobiliary duct geometry. The segmenting may include smoothing and denoising. The computing may include applying incompressible Navier-Stokes equations. The at least one flow rate value may include a plurality of flow rate values, and the computing the relative pressure distribution along the pancreaticobiliary duct geometry may be based on the plurality of flow rate values and the pancreaticobiliary duct geometry.

According to various embodiments, a system for non-invasively assessing pancreaticobiliary duct hypertension is presented. The system includes: a non-transitory computer readable medium comprising instructions; and at least one electronic processor that executes the instructions to perform operations comprising: obtaining a three-dimensional image of a patient's pancreaticobiliary duct; segmenting the three-dimensional image of the patient's pancreaticobiliary duct, from which a pancreaticobiliary duct geometry is obtained; specifying at least one flow rate value for the patient; simulating, based on the at least one flow rate value and the pancreaticobiliary duct geometry, a relative pressure distribution along the pancreaticobiliary duct geometry; determining, based at least on the relative pressure distribution, a pancreaticobiliary duct hypertension quantification for the patient; and providing the pancreaticobiliary duct hypertension quantification for the patient.

Various optional features of the above system embodiments include the following. The pancreaticobiliary duct geometry may include a pancreatic duct geometry, the flow rate value may include a pancreatic juice flow rate value, and the pancreaticobiliary duct hypertension quantification may include a pancreatic duct hypertension quantification. The operations may include predicting, based on the pancreatic duct hypertension quantification for the patient, a patient response to pancreatic duct hypertension relief. One of pancreatic duct endotherapy or pancreatic duct surgery may be performed on the patient based on the predicting. The three-dimensional image may include an MRI image. The pancreaticobiliary duct hypertension quantification for the patient may include an estimated pressure difference between two locations within the patient's pancreaticobiliary duct. The pancreaticobiliary duct hypertension quantification for the patient may include an estimated pancreaticobiliary duct resistance. The determining may include performing a regression for a plurality of flow rate values and a corresponding plurality of pressure difference values for the pancreaticobiliary duct geometry. The segmenting may include smoothing and denoising. The computing may include applying incompressible Navier-Stokes equations. The at least one flow rate value may include a plurality of flow rate values, and the computing the relative pressure distribution along the pancreaticobiliary duct geometry may be based on the plurality of flow rate values and the pancreaticobiliary duct geometry.

Combinations, (including multiple dependent combinations) of the above-described elements and those within the specification have been contemplated by the inventors and may be made, except where otherwise indicated or where contradictory.

### BRIEF DESCRIPTION OF THE DRAWINGS

Various features of the examples can be more fully appreciated, as the same become better understood with reference to the following detailed description of the examples when considered in connection with the accompanying figures, in which:

FIG. 1 is an anatomical diagram illustrating a pancreatic duct and a bile duct according to various embodiments;

FIG. 2 depicts a magnetic resonance cholangiopancreatography (MRCP) image and a corresponding pancreatic duct geometry, according to various embodiments;

FIG. 3 illustrates relative pressure distributions along a pancreaticobiliary duct geometry for three different flow rates, according to various embodiments;

FIG. 4 is a flow diagram for a method of non-invasively assessing pancreaticobiliary duct hypertension, according to various embodiments;

FIG. 5 shows a graph illustrating a correlation between pressure estimates by a reduction to practice and empirical in-vivo pressure measurements; and

FIG. 6 shows a graph illustrating correlation between pressure drops estimated by a reduction to practice and patient pain relief scores, for a mass flow rate of 0.1 g/s.

### DESCRIPTION OF THE EXAMPLES

Reference will now be made in detail to example implementations, illustrated in the accompanying drawings. Wherever convenient, the same reference numbers will be used throughout the drawings to refer to the same or like parts. In the following description, reference is made to the accompanying drawings that form a part thereof, and in which is shown by way of illustration specific exemplary examples in which the invention may be practiced. These examples are described in sufficient detail to enable those skilled in the art to practice the invention and it is to be understood that other examples may be utilized and that changes may be made without departing from the scope of the invention. The following description is, therefore, merely exemplary.

Accurately measuring bile duct or pancreatic duct pressure may help in deciding if an intervention is going to benefit the patient. Over 700,000 ERCPs are performed in the U.S. alone, and around 35% are diagnostic or relevant to pathologies that may involve an unclear degree of obstruction. Non-limiting examples of pathologies that involve an unclear degree of ductal obstruction or ductal hypertension include chronic pancreatitis, pancreas divisum, sphincter of oddi dysfunction, cystic fibrosis-related disorders in the pancreas, mucinous pancreatic cysts communicating with the pancreas duct, etc.

Some embodiments provide an imaging-based non-invasive methodology to measure the pressure within a patient's bile duct and/or pancreatic duct, which may be used to evaluate the presence of ductal hypertension and direct management. Some embodiments utilize magnetic resonance imaging (MRI)-based real-time measurement of pressure within a patient's bile duct and/or pancreatic duct. Some embodiments utilize MRI image processing to determine pressures within a patient's bile duct and/or pancreatic duct. Some embodiments non-invasively identify if there is true ductal hypertension and can thus infer the presence of an obstructive pathology that is impacting flow. Some embodiments can therefore be used to direct management and follow-up for the patient.

These and other features and advantages are shown and described herein in reference to the figures. Note that embodiments are shown and described herein in reference to chronic pancreatitis by way of non-limiting example. However, embodiments may be used to assess pancreaticobiliary duct hypertension in general, including in the bile duct, and not limited to the pancreatic duct. Thus, embodiments may be used to assess whether invasive interventions may be successful to relieve hypertension in any pancreaticobiliary duct.

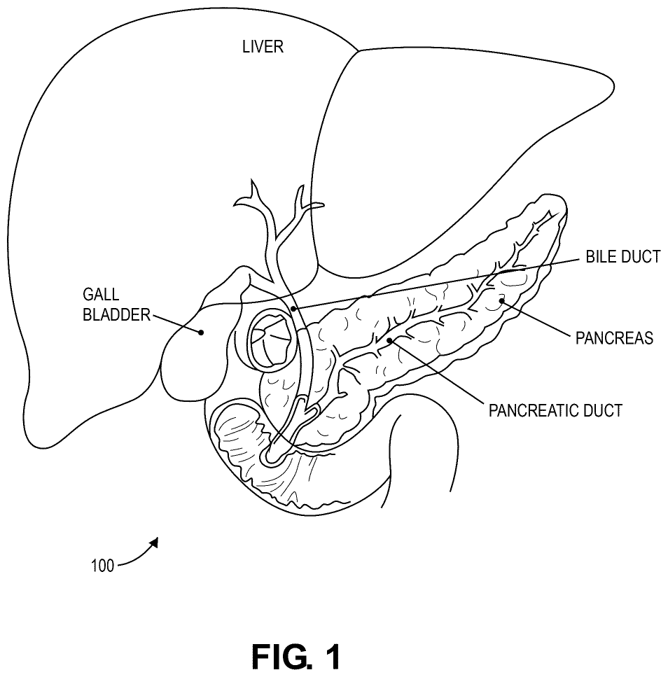

FIG. 1 is an anatomical diagram 100 illustrating a pancreatic duct and a bile duct according to various embodiments. As shown, the pancreatic duct conveys pancreatic juice produced by the pancreas to the duodenum. As shown in FIG. 1, the bile duct conveys bile from the gall bladder and other fluids from the liver to the duodenum. In general, embodiments may be used to determine pressure or otherwise assess hypertension in any pancreaticobiliary duct, such as a pancreatic duct and/or a bile duct.

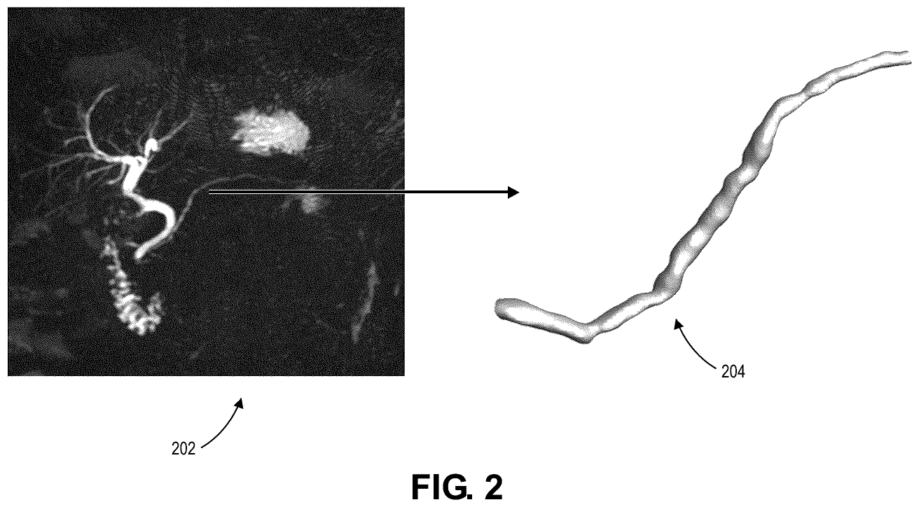

FIG. 2 depicts a magnetic resonance cholangiopancreatography (MRCP) image 202 and a corresponding pancreatic duct geometry 204, according to various embodiments. In general, embodiments may utilize an MRCP image together with one or more specified flow rates to determine a pancreaticobiliary duct pressure or otherwise assess pancreaticobiliary duct hypertension. To do so, embodiments may utilize an MRCP image, such as the MRCP image of the pancreatic duct 202 shown in FIG. 2, acquired from a patient using an MRI system. In general, the MRCP image may be in three dimensions and may depict any pancreaticobiliary duct of the patient. The MRCP image may be high-resolution, e.g., with 1 mm×1 mm voxels in 3 mm slices. Embodiments may automatically segment the MRCP image, which may include denoising and/or smoothing, to generate a three-dimensional pancreaticobiliary duct geometry, e.g., the pancreatic duct geometry 204 shown in FIG. 2. According to some embodiments, segmentation may be achieved using MIMICS software, available from Materialise NV, of Belgium. Thus, some embodiments generate a patient-specific three-dimensional pancreaticobiliary duct geometry from a non-invasive MRCP image.

Small branches of the pancreatic duct may not be well resolved by even the highest-resolution MRI. As a result, one or more thin branches remain undetected. Therefore, some embodiments account for the effect of such missing branches on ductal flow as mass sources applied at the duct boundaries.

Note that although embodiments are described herein in reference to MRCP images, embodiments are not so limited. For example, some embodiments may utilize a three-dimensional computer tomography (CT) image of a patient pancreaticobiliary duct. Such embodiments may segment the CT image to obtain a three-dimensional pancreaticobiliary duct geometry as described herein.

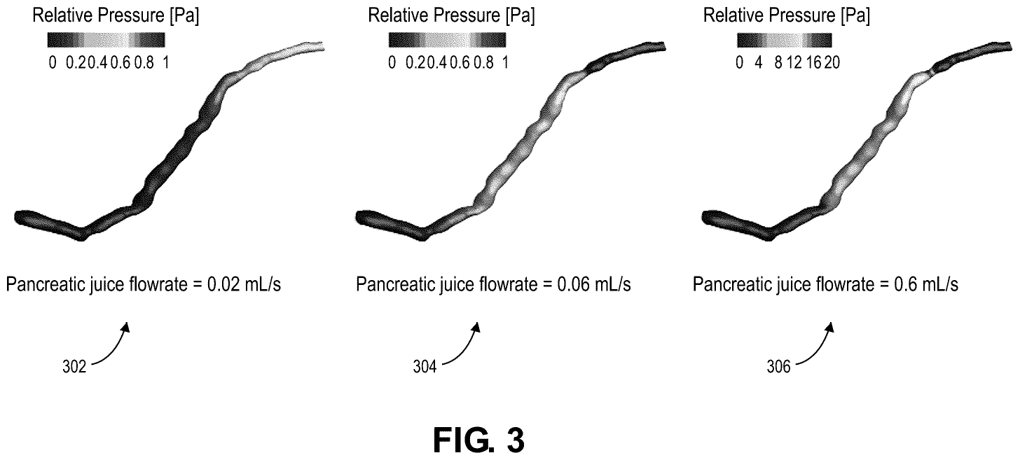

FIG. 3 illustrates relative pressure distributions 302, 304, 306 along a pancreaticobiliary duct geometry for three different flow rates, according to various embodiments. In general, embodiments may model one or more specified flow rates in a three-dimensional pancreaticobiliary duct geometry, such as is shown and described herein in reference to FIG. 2, to determine a pancreaticobiliary duct pressure or otherwise assess pancreaticobiliary duct hypertension. According to some embodiments, the one or more flow rates for pancreatic juice may be specified directly, e.g., as one or more values in the range of 0.05 to 0.5 mL/s, e.g., with a physiological mean value of about 0.1 mL/s. According to some embodiments, the one or more flow rates for pancreatic juice may encompass a broader range of physiological conditions, e.g., any flow rate from 0.1 to 5 mL/min (approximately 0.002 to 0.1 mL/s). According to some embodiments, the mass sources used to account for missing branches may be specified as, by way of non-limiting example, 0.01, 0.05, 0.1, or 0.2 g/s.

The specified flow rates according to various embodiments may be based on flow rates that are characteristic for a variety of conditions, which may be reproduced in a patient when the three-dimensional image of the patient's pancreaticobiliary duct is obtained. For example, some embodiments may specify a pancreatic juice flow rate that is characteristic of a human after having been injected with secretin. As another example, some embodiments may specify a pancreatic juice flow rate that is characteristic of a pancreatic juice flow rate in a human at a given time interval after eating.

Some embodiments may specify flow rate values according to values determined empirically using porcine models. According to a non-limiting example embodiment, a porcine pancreas may be obtained, the pancreatic duct ligated on one side, and contrast injected into the pancreatic duct on the other side using a balloon catheter. The pancreas may be imaged using high-resolution CT to characterize the detailed pancreatic ductal anatomy and characterize the ductal flow to develop a fluid dynamics-based pressure estimation. Similar determinations may be performed for porcine bile flow rates for embodiments that quantify bile duct hypertension.

Some embodiments impose a Dirichlet boundary condition with zero pressure at the outlet of the pancreatic duct. This condition helps to ensure that the outlet serves as a pressure reference point, facilitating accurate simulation of fluid flow dynamics within the pancreatic duct simulation.

For each specified flow rate, some embodiments model a respective relative pressure distribution within the three-dimensional pancreaticobiliary geometry for the patient. Each relative pressure distribution may specify relative pressure values at a variety of locations within the pancreaticobiliary duct geometry. As shown in FIG. 3, three relative pressure distributions 302, 304, 306 for a particular pancreatic duct geometry are shown for pancreatic juice flow rates of 0.02, 0.06, and 0.6 mL/s, respectively. According to some embodiments, a relative pressure distribution along the pancreaticobiliary duct geometry may be computed by solving incompressible Navier-Stokes equations computationally, e.g., using a computer simulation. For incompressible Navier-Stokes equation discretized in three-dimensional Cartesian grid, the governing equations may be represented as follows, by way of non-limiting example, where u is fluid velocity vector, p is the pressure, and p and p are density and dynamic viscosity of pancreatic juice:

$$Δ \cdot u = 0$$

$$ρ \frac{\partial u}{\partial t} + ρ ( u \cdot ∇ ) u + ∇ p = μ {∇}^{2} u$$

Embodiments may model the fluid flow and/or pressure based on laminar flow with the characteristics (e.g., viscosity) of water.

Embodiments may determine a pancreaticobiliary duct hypertension quantification for a patient based on one or more pancreaticobiliary duct relative pressure distributions corresponding to one or more specified flow rates. Non-limiting examples of such determinations follow.

According to some embodiments, the pancreaticobiliary duct hypertension quantification may be determined as a difference between pressures at proximal and distal ends of the pancreaticobiliary duct for a given relative pressure distribution. For example, for the relative pressure distribution 304 shown in FIG. 3, the proximal relative pressure is about 0.1 Pa and the distal relative pressure is about 0.9 Pa, for a difference in pressure of 0.8 Pa. An average of values for such differences over a plurality of flow rates may be used according to some embodiment.

According to some embodiments, the pancreaticobiliary duct hypertension quantification may be determined as a pancreaticobiliary duct resistance value. In general, the resistance may be defined as, by way of non-limiting example, R=ΔP/Q, where R is the resistance value, ΔP is the relative pressure difference, e.g., between pressures at proximal and distal ends of the pancreaticobiliary duct, and Q is the flow rate. Because R is a proportion, according to some embodiments, R may be determined by a linear regression relating ΔP and Q for a plurality of values of Q. For example, according to such embodiments, the resistance R may be determined as the slope of the line interpolated by such a regression. According to some embodiments, the resistance R may be represented by R=Rᵥ+RₗQ, where Rᵥ is the vicious, friction resistance, and Rₗ is the inertial resistance, and Q is the flow rate. Values for Rᵥ and Rₗ may be obtained from a computational fluid dynamics simulation results, e.g., by solving incompressible Navier-Stokes equations.

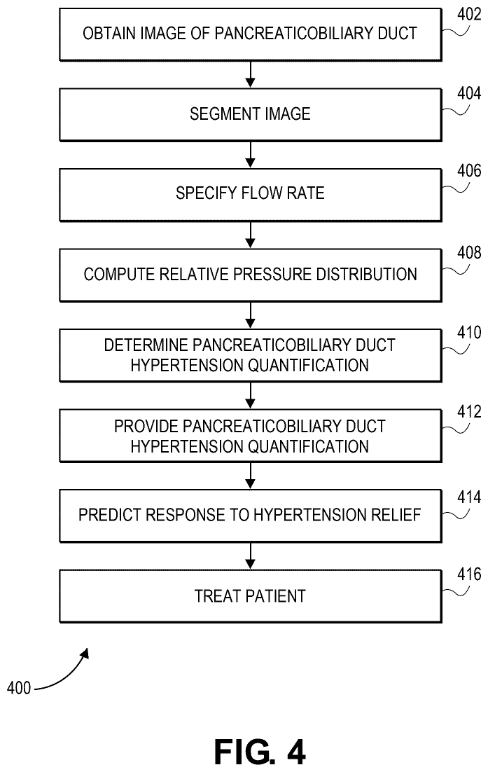

FIG. 4 is a flow diagram 400 for a method of non-invasively assessing pancreaticobiliary duct hypertension, according to various embodiments. The method 400 may be performed using a 3D imaging system, such as an MRI or CT scanner, and a computing device, e.g., an electronic processor internal to the MRI or CT scanner system, or a desktop computer. The method 400 may be used to assess pancreatic duct hypertension, bile duct hypertension, or a different pancreaticobiliary duct hypertension.

At 402, the method 400 includes obtaining a three-dimensional image of a patient's pancreaticobiliary duct. According to some embodiments, the three-dimensional image may be an MRI image, e.g., an MRCP image. According to some embodiments, the three-dimensional image may be a CT image. Other three-dimensional images may be used according to various embodiments.

At 404, the method 400 includes segmenting the three-dimensional image of the patient's pancreaticobiliary duct. The segmenting may include denoising and/or smoothing, for example. The segmenting may be performed automatically, e.g., using segmentation software operating on the three-dimensional image. The segmenting produces a three-dimensional pancreaticobiliary duct geometry, which may be represented in a computer in any of a variety of formats that preserve three-dimensional relationships.

At 406, the method 400 includes specifying at least one flow rate value for the patient. The flow rate value(s) may be for pancreatic juice, bile, or any other pancreaticobiliary fluid. One or more values may be specified. The value(s) may be previously determined as described herein in reference to FIG. 3, for example. The value(s) may be specified as parameter(s) for a computer-implemented computational fluid dynamics simulation, for example, as shown and described herein in reference to FIG. 3.

At 408, the method 400 includes simulating, based on the at least one flow rate value and the pancreaticobiliary duct geometry, a relative pressure distribution along the pancreaticobiliary duct geometry. The relative pressure distribution may be computed as shown and described herein in reference to FIG. 3, for example.

At 410, the method 400 includes determining, based at least on the relative pressure distribution, a pancreaticobiliary duct hypertension quantification for the patient. The pancreaticobiliary hypertension qualification may be in the form of the relative pressure difference itself, a resistance, or a hypertension different quantification, e.g., as described herein in reference to FIG. 3.

At 412, the method 400 includes providing the pancreaticobiliary duct hypertension quantification for the patient. The method 400 may provide the pancreaticobiliary duct hypertension quantification by outputting from a computer, e.g., in visual form on a monitor, to an electronic medical records system, over a network, or into persistent storage, according to various non-limiting example embodiments.

At 414, the method 400 includes predicting, based on the pancreatic duct hypertension quantification for the patient, a patient response to pancreatic duct hypertension relief. For example, the prediction may be based on whether the pancreaticobiliary duct hypertension quantification is indicative of hypertension, e.g., by exceeding a specified threshold.

At 416, the method 400 includes treating the patient based on the prediction of 414. The treatment may be invasive, according to some embodiments. By way of non-limiting example, the treatment may include performing one of pancreatic duct endotherapy or pancreatic duct surgery on the patient, or otherwise relieving pancreaticobiliary hypertension in the patient. This may conclude the method 400.

A usage and validation of a reduction to practice is described presently. As part of the reduction to practice validation, 27 chronic pancreatitis patients underwent MRCP with or without the use of secretin, and pancreatic duct pressure parameters were non-invasively determined using the reduction to practice. The reduction to practice included determining the relative pressure distribution along the pancreatic duct from simulation results, as shown and described herein in reference to FIG. 3. Subsequently, to validate the pancreaticobiliary hypertension quantification values determined by the reduction to practice, the patients underwent clinically indicated invasive ERCP, during which a cardiac pressure sensor guidewire was placed into the pancreatic duct, and direct measurement of the pancreatic duct pressure was performed. A strong correlation was found between the pancreatic duct pressures determined by the reduction to practice and the direct pancreatic duct pressure measurement using ERCP (R²=0.70). Notably, patients with severe pain had a higher mean pancreatic duct pressure compared to patients with mild symptoms (19.1 mm Hg vs 7.3 mm Hg, p=0.04).

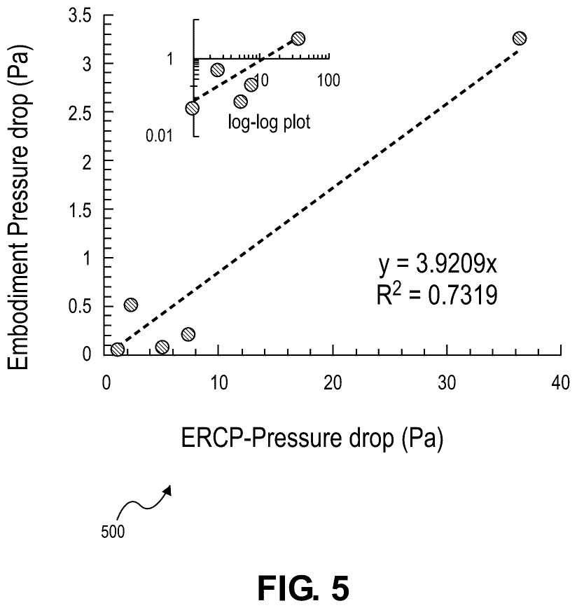

FIG. 5 shows a graph 500 illustrating a correlation between pressure estimates by a reduction to practice and empirical in-vivo pressure measurements. The x-axis units Pa, from a measured pressure drop from ERCP, and the y-axis units are Pa, with a total mass source of 0.01 g/s. In particular, the x-axis data depicted by the graph 500 represent clinical measurements of pressure drop across the pancreatic duct in patients undergoing clinically indicated ERCP/endotherapy, including pancreatic duct stenting as needed to relieve ductal obstruction, and the y-axis represents estimates from a reduction to practice for the same patients.

The results illustrated by FIG. 5 demonstrate that the high-pressure regions identified by the reduction to practice correspond well with the clinical measurements. The consistent agreement between simulation and clinical data validates the reduction to practice and highlights the applicability of various embodiments to clinical practice.

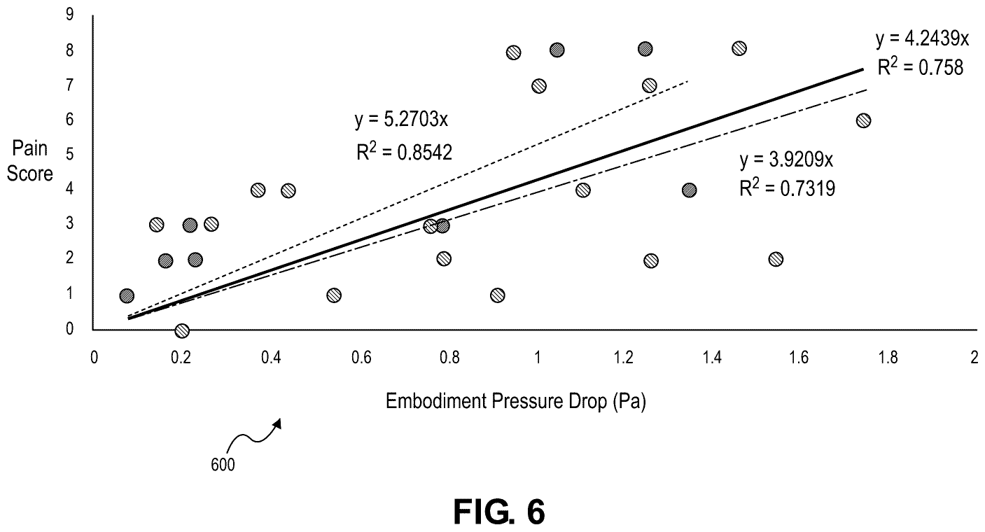

Further validation is provided by comparing pressure drops predicted by the reduction to practice with clinical patient pain relief reports, as shown and described presently in reference to FIG. 6.

FIG. 6 shows a graph 600 illustrating correlation between pressure drops estimated by the reduction to practice and patient pain relief scores, for a mass flow rate of 0.1 g/s. In particular, the predicted pressure drops were compared to scores representing the difference in pain levels reported by patients before and after surgery, represented as Pain_{ref}=Pain_{before}−Pain_{after}. Data points from two independently-acquired patient groups are shown separately in the graph 600, represented as light and dark grey, as well as combined. For each group, pain scores before and after surgery were measured, the corresponding pain-relief score was computed, and the associated simulated pressure drop was determined. As shown in the graph 600, the correlation between the pain-relief scores and the predicted pressure drop was high, with R² values ranging from 0.73 to 0.85. For the combined dataset, the R² value was 0.758.

This strong correlation indicates that the patients with higher simulated pressure drops tended to experience greater pain relief, suggesting that pancreatic duct hypertension was the primary cause of their pain. Because pancreatic ductal hypertension—rather than neuropathy, which is less responsive to surgical intervention—is specifically reflected in the pain-relief score, the correlation between our simulated pressure drop and the pain-relief score serves as a crucial indicator of the efficacy of the reduction to practice.

Further analysis, where the pressure drop was normalized by the length of the pancreatic duct, revealed a quadratic relationship between the pressure drop and the total mass source. This further supports the validity of the reduction to practice in capturing the physiological mechanisms underlying pain relief.

Thus, techniques for non-invasively assessing pancreaticobiliary duct hypertension are disclosed herein. According to some use cases, the imaging-based pancreatic duct and bile duct pressure measurement technology can be implemented alongside standard MRI as an additional imaging protocol. It is expected that gastroenterologists, endoscopists, and surgeons will benefit from the disclosed technology, which may be used to guide management and follow-up. Further, MRI manufacturers may implement the disclosed imaging protocol and processing. Yet further, the disclosed MRI-based pressure measurement technique may eventually replace around 200,000-250,000 annual ERCPs in the U.S. currently being performed. Embodiments may have additional applications, such as in the liver and portal vein pressure measurement to assist in the management of various liver pathologies.

Certain examples can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), flash memory, and magnetic or optical disks or tapes.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented using computer readable program instructions that are executed by an electronic processor.

These computer readable program instructions may be provided to a processor of a general-purpose computer, special purpose computer, or other programmable data processing apparatus to produce a machine, such that the instructions, which execute via the electronic processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the C programming language or similar programming languages. The computer readable program instructions may execute entirely on a user's computer, partly on the user's computer, as a stand-alone software package, partly on the user's computer and partly on a remote computer or entirely on the remote computer or server.

As used herein, the terms “A or B” and “A and/or B” are intended to encompass A, B, or {A and B}. Further, the terms “A, B, or C” and “A, B, and/or C” are intended to encompass single items, pairs of items, or all items, that is, all of: A, B, C, {A and B}, {A and C}, {B and C}, and {A and B and C}. The term “or” as used herein means “and/or.”

As used herein, language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” is intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. § 112(f). However, for any claims containing elements designated in any other manner, it is intended that such elements are not to be interpreted under 35 U.S.C. § 112(f).

While the invention has been described with reference to the exemplary examples thereof, those skilled in the art will be able to make various modifications to the described examples without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A method of non-invasively assessing pancreaticobiliary duct hypertension, the method comprising:
obtaining a three-dimensional image of a patient's pancreaticobiliary duct;
segmenting the three-dimensional image of the patient's pancreaticobiliary duct, from which a pancreaticobiliary duct geometry is obtained;
specifying at least one flow rate value for the patient;
simulating, based on the at least one flow rate value and the pancreaticobiliary duct geometry, a relative pressure distribution along the pancreaticobiliary duct geometry;
determining, based at least on the relative pressure distribution, a pancreaticobiliary duct hypertension quantification for the patient; and
providing the pancreaticobiliary duct hypertension quantification for the patient.

2. The method of claim 1, wherein the pancreaticobiliary duct geometry comprises a pancreatic duct geometry, wherein the flow rate value comprises a pancreatic juice flow rate value, and wherein the pancreaticobiliary duct hypertension quantification comprises a pancreatic duct hypertension quantification.

3. The method of claim 2, further comprising predicting, based on the pancreatic duct hypertension quantification for the patient, a patient response to pancreatic duct hypertension relief.

4. The method of claim 3, further comprising performing one of pancreatic duct endotherapy or pancreatic duct surgery on the patient based on the predicting.

5. The method of claim 1, wherein the three-dimensional image comprises an MRI image.

6. The method of claim 1, wherein the pancreaticobiliary duct hypertension quantification for the patient comprises an estimated pressure difference between two locations within the patient's pancreaticobiliary duct.

7. The method of claim 1, wherein the pancreaticobiliary duct hypertension quantification for the patient comprises an estimated pancreaticobiliary duct resistance.

8. The method of claim 7, wherein the determining comprises performing a regression for a plurality of flow rate values and a corresponding plurality of pressure difference values for the pancreaticobiliary duct geometry.

9. The method of claim 1, wherein the segmenting comprises smoothing and denoising.

10. The method of claim 1, wherein the computing comprises applying incompressible Navier-Stokes equations.

11. The method of claim 1, wherein the at least one flow rate value comprises a plurality of flow rate values, and wherein the computing the relative pressure distribution along the pancreaticobiliary duct geometry is based on the plurality of flow rate values and the pancreaticobiliary duct geometry.

12. A system for non-invasively assessing pancreaticobiliary duct hypertension, the system comprising: a non-transitory computer readable medium comprising instructions; and at least one electronic processor that executes the instructions to perform operations comprising:
obtaining a three-dimensional image of a patient's pancreaticobiliary duct;
segmenting the three-dimensional image of the patient's pancreaticobiliary duct, from which a pancreaticobiliary duct geometry is obtained;
specifying at least one flow rate value for the patient;
simulating, based on the at least one flow rate value and the pancreaticobiliary duct geometry, a relative pressure distribution along the pancreaticobiliary duct geometry;
determining, based at least on the relative pressure distribution, a pancreaticobiliary duct hypertension quantification for the patient; and
providing the pancreaticobiliary duct hypertension quantification for the patient.

13. The system of claim 12, wherein the pancreaticobiliary duct geometry comprises a pancreatic duct geometry, wherein the flow rate value comprises a pancreatic juice flow rate value, and wherein the pancreaticobiliary duct hypertension quantification comprises a pancreatic duct hypertension quantification.

14. The system of claim 13, wherein the operations further comprise predicting, based on the pancreatic duct hypertension quantification for the patient, a patient response to pancreatic duct hypertension relief.

15. The system of claim 14, wherein one of pancreatic duct endotherapy or pancreatic duct surgery are performed on the patient based on the predicting.

16. The system of claim 12, wherein the three-dimensional image comprises an MRI image.

17. The system of claim 12, wherein the pancreaticobiliary duct hypertension quantification for the patient comprises an estimated pressure difference between two locations within the patient's pancreaticobiliary duct.

18. The system of claim 12, wherein the pancreaticobiliary duct hypertension quantification for the patient comprises an estimated pancreaticobiliary duct resistance.

19. The system of claim 18, wherein the determining comprises performing a regression for a plurality of flow rate values and a corresponding plurality of pressure difference values for the pancreaticobiliary duct geometry.

20. The system of claim 12, wherein the segmenting comprises smoothing and denoising.

21. The system of claim 12, wherein the computing comprises applying incompressible Navier-Stokes equations.

22. The system of claim 12, wherein the at least one flow rate value comprises a plurality of flow rate values, and wherein the computing the relative pressure distribution along the pancreaticobiliary duct geometry is based on the plurality of flow rate values and the pancreaticobiliary duct geometry.
